(19) United States
(12) Patent Application Publication
(10) Pub. No.: US 2011/0296728 A1
(43) Pub. Date: Dec. 8, 2011

(54) USER CONFIGURABLE IDENTIFICATION APPARATUS AND METHOD

(76) Inventor: Steve Bolt, Fallbrook, CA (US)

(21) Appl. No.: 13/152,176

(22) Filed: Jun. 2, 2011

Related U.S. Application Data
(60) Provisional application No. 61/350,869, filed on Jun. 2, 2010.

(51) Int. Cl. G09F 3/18; G06K 19/06; A61B 5/117

(52) U.S. Cl. 40/649; 235/494

(57) ABSTRACT

A user assembleable identification device employable as a dog tag or other user-worn identifier configured for wearing around the neck on a chain or similar restraint. The device is provided in a kit of components assembleable to a readable tag which all come engaged to a chain or similar restraint to prevent component loss during transport. The assembled dog tag is formed of a malleable frame having a window therein surrounded by a slot. A cover sheet provides a templet for the user to cut a user formed display sheet to size. The display sheet bears user imparted identification indicia and is sandwiched between two protective cover sheets and engaged in the slot. Instructions are placed on the cover sheet for the user to assemble the device.

This application claims priority to U.S. Provisional Patent Application No. 61/350,869 filed on Jun. 2, 2011. The invention herein disclosed relates generally to the art of identification tags. More particularly, it relates to a device and method adapted for easy user configuration and assembly of wearable identification device, similar in appearance to “dog tags” which are worn by military personnel and other individuals where identification is a paramount requirement, especially where the user is unable to respond to inquiry as to their identity.

### BACKGROUND OF THE INVENTION

1. Field of the Invention

2. Prior Art

Identification devices worn by individuals have been employed for many years. One such device which in the last century has obtained notoriety is the “dog tag.” The term dog tag is an informal name for what are essentially identification tags worn by military personnel. It is thought the term has come about due to their resemblance to actual dog tags. Such conventional tag components are primarily employed for the identification of dead and wounded personnel and allow medical and other personnel to ascertain basic identification and medical information of the individual wearing the tags which may be employed for medical treatment of the wearer. Conventionally, such tags contain information about critical medical data such as blood type [1] and history of inoculations and perhaps allergies. This minimal amount of information is generally required by medical personnel to render initial medical assistance in the field.

Conventional dog tags are generally fabricated from a corrosion-resistant metal or alloy such as aluminum or stainless-steel. This is to allow them to remain intact when exposed to corrosive environments. In the event the dog tag wearer has a medical condition that requires special attention, an additional tag with the pertinent medical or other information has been conventionally issued and worn with the other dog tags. In non military situations such as students at schools or other organizations wishing to provide individual identification and/or medical information, similar devices to dog tags for the military are employed.

However, conventional metallic dog tags or identification and information tags worn around the neck, must be manufactured using machinery such as metal presses. Essentially lettering is pressed into the planar metal material causing a depression on one side and raised lettering on the opposite side of the metal sheeting. While permanent in nature, such a process severely limits the amount of information that may be imparted to the tags. Further, such a process requiring heavy press machinery limits where the tags may be manufactured, and severely limits subsequent alteration of the tag's information since once the wearer becomes distant from the heavy machine required to make or change the tag, it is unlikely they will be able to get the information changed or new tags issued. This is especially so in the miliary where generally once a solder is issued clothing or other items, they rarely are able to get such re-issued at a later date.

Consequently, identification and information tags worn by individuals contain little information whereby the individual wearing them may be visually matched to the tags or identified facially. Further, as noted such tags easily become outdated due to the lack of access to machinery to reform them or make new tags. Additionally, the metal pressing process severely limits the amount of information that may be imparted to the planar metal component since the very process deforms the metal and too many letters or numbers will begin to deform each other and render the information indiscernible.

As such, there exists an unmet need, for wearable identification tags for individuals which may be formed to contain wearer data, and easily amended at a later date should that data change. Such a device should allow for wearer or user configuration, thereby allowing the user, who has the most to lose or gain from the information imparted to the tags, to form them and amend them. Further, such a device should not only provide means to impart lettering and numbering indicia to the tag formed, it should also allow for photographic information to be imparted to the assembled tag such that the tags may contain the face of the wearer and allow for user identification by simply viewing. Viewable photos of the wearer provides for a quicker matching of the tags to the wearer and lessens the chance that reading errors can cause mis-identification. Finally, such an identification tag system should protect the indicia, and photographic indicia thereon to render them usable after being subjected to wet or other harsh environments over time.

With respect to the above, before explaining at least one preferred embodiment of the user configurable identification tag system and method herein in detail or in general, it is to be understood that the disclosed invention is not limited in its application and to the details of construction and to the arrangement of the assembled or other components or the steps set forth in assembly in the following description or illustrated in the drawings. The various apparatus and methods of the disclosed user configurable identification tags are capable of other embodiments, and of being practiced and carried out in various ways, all of which will be obvious to those skilled in the art, once the information herein is reviewed. Also, it is to be understood that the phraseology and terminology employed herein are for the purpose of description to those skilled in the are and should not be regarded as limiting.

As such, those skilled in the art will appreciate that the conception upon which this disclosure is based, may readily be utilized as a basis for designing other modes of forming and deploying a user assembleable identification system and for carrying out the several purposes of the present disclosed device and method. It is important, therefore, that the embodiments, objects and claims herein, be regarded as including such equivalent construction and methodology, insofar as they do not depart from the spirit and scope of the present invention.

### SUMMARY OF THE INVENTION

The disclosed device and method provide a solution to the above noted shortcomings of conventional personnel identification tags or dog tags which is done in such a manner as to improve on the amount and type of information the tags may carry, and in allowing for changes to be made to such, subsequent to the initial formation of the tags.

In one preferred mode of the device, a kit is provided for the user to assemble their own identification tag. The kit provides a rubber or otherwise malleable perimeter edge component, a pair of adhesive backed transparent sheets, a protective plastic sheet, a substantially rigid base, and instructions for assembly of the finished device.

All components provided are adapted for engagement on a chain or tether to thereby maintain them in proximity to each other and ready to use. Additionally, the malleable perimeter edge component provides a means to prevent the device from making noise as well as to protect the user's skin from the edges of the layered component it holds together. The user need only provide a photograph if that is what is desired, or may provide indicia including name, blood type, allergies, a bar code for medical records or a medical record number, and a photo, or decorative indicia, or a combination of a photo and identification and/or medical indicia for enclosure in the device. If a barcode or medical record ID number is included, which is preferred, a medic or medical personnel with a PDA or computer pad or other networked device, can enter the code or bar and download the wearer's medical records.

In use, the user will read the provided instructions engaged with the kit upon the chain. In a first step, the malleable perimeter edge is removed from a biased engagement on a planar rear base component. In a second step, the user employs the clear plastic planar sheet as a template to trim a photo of themselves to the correct size. Next, the user stacks the components to assemble the device by placing a first of the adhesive backed sheets adjacent to the planar rear base. Then the trimmed photo is stacked on the adhesive backed sheet whereafter a second of the adhesive sheets is engaged to the photo. In another step the clear plastic planar sheet is stacked on top of the second of the adhesive backed sheets. Finally, the malleable perimeter edge is engaged around the perimeter of the stacked components by engaging their stacked perimeter in a formed recess within the malleable perimeter edge component. The assembled product is thereafter engaged to a chain and is rendered durable and water resistant by the biased engagement of the malleable perimeter like a seal around the perimeter of the stacked components.

Indicia may be imparted to the front or rear of the photo or the rear of the planar base component. Alternatively, indicia may be imparted to the front of the planar base and would be legible if the device is disassembled along with any indicia on the rear of the photo. Such indicia may be medical records, identification information or other information personal to the user.

It is an object of this invention to provide a user assembleable identification device that may be worn on their person.

It is another object of this invention to provide the device in a kit of components the user may assemble.

A further object is the provision of one or both of a photo rendition of the wearer and informational indicia.

These together with other objects and advantages which become subsequently apparent reside in the details of the identification tag system and method herein as more fully hereinafter described and claimed, reference being had to the accompanying drawings forming a part thereof, wherein like numerals refer to like parts throughout.

### BRIEF DESCRIPTION OF DRAWING FIGURE

FIG. 1 depicts a favored mode of the device provided in a kit form with all components configured to engage upon a chain as a means to keep kit parts together until employed.

FIG. 2 shows another view of the device of FIG. 1 after assembly by the user and inclusion of a photo.

FIG. 3 shows a first side of the base component having indicia thereon.

FIG. 4 depicts an opposite side of the base component showing indicia placed thereon.

FIG. 5 depicts a transparent cover sheet engageable within the malleable perimeter edge component of FIG. 9.

FIG. 6 shows the pair of adhesive backed transparent sheets adapted to cover a trimmed photo of FIG. 9.

FIG. 7 show the instructions included in the kit of FIG. 1.

FIG. 8 shows the photo or indicia such as a barcode or medical record number of the user trimmed using the transparent cover sheet of FIG. 5 as a templet and showing the indicia indicating template placed where it will be covered when assembled.

FIG. 9 depicts the malleable perimeter of the device having an interior opening with a recess formed along the interior wall of the perimeter of the opening configured to overlay and surround the assembled components in a biased waterproof engagement and hide instructional indicia.

### DETAILED DESCRIPTION OF THE PREFERRED EMBODIMENTS OF THE INVENTION

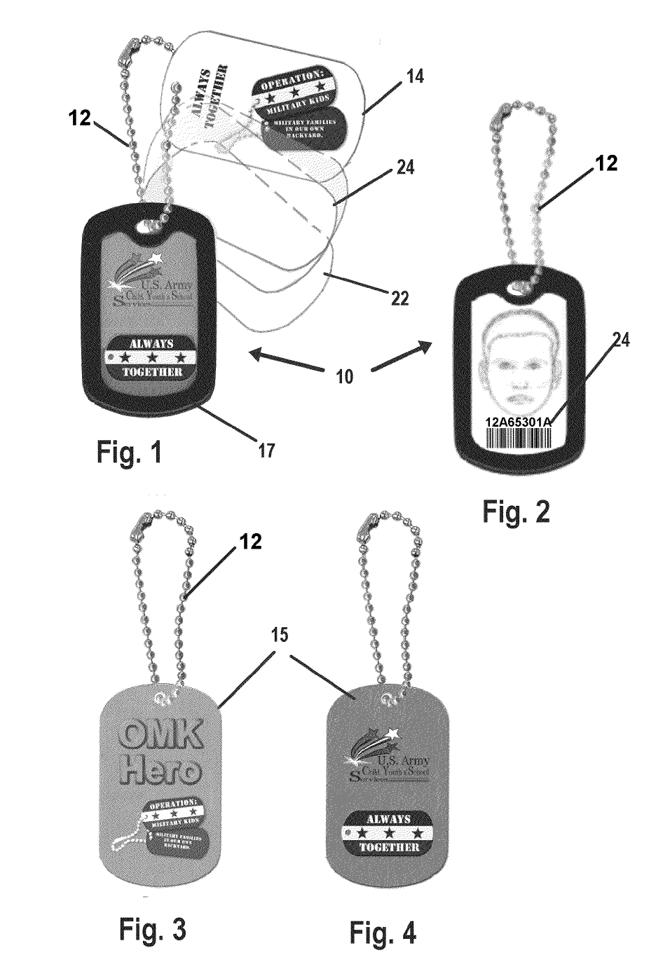

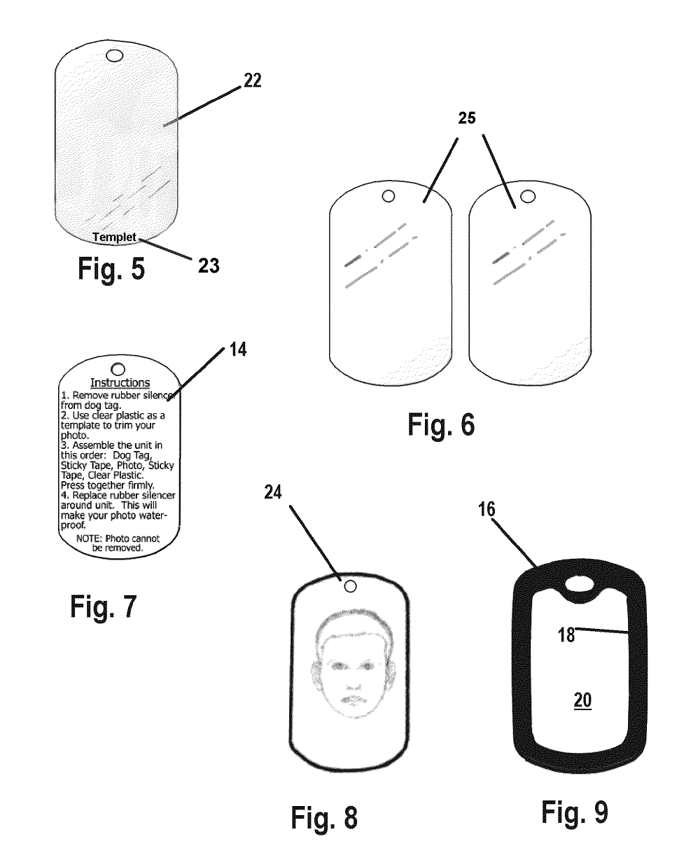

Referring now to the FIGS. 1-9, the user configurable identification apparatus provided in the kit form of FIG. 1 where all or some of the components may be custom assembled to form the device in an as-used mode. The components are provided engaged upon a chain 12 to maintain them in a kit where components cannot be lost prior to assembly. In addition to the components, the device 10 is assembleable in the method of the instructions 14 of FIG. 7 placed on the cover sheet 22, to yield the assembled device 10 of FIG. 2 in the as-used mode.

The user will employ the kit of FIG. 1 with all components engaged by a chain 12 to maintain the kit from losing parts. The user will remove the chain 12 to separate the various components for assembly in an individually customized fashion to the as-used mode of FIG. 2 for instance.

In a first step of forming the device 10 in an as-used user-customized mode, the malleable edge 16 of FIG. 9 is removed to allow separation of the components which are engageable in a slot 18 formed around the interior edge of the perimeter of an aperture 20 of the edge 16.

Using the transparent cover sheet 22, which has indicia 23 thereon identifying it, which is positioned to be covered and out of sight when in the as-used mode, a templet for sizing a photo 24 is provided to allow the photo 24 to be trimmed to fit properly in the window 20 and to have a properly sized perimeter to slide into the biased engagement of the flexible material forming the slot 18.

Two adhesive backed transparent sheets 25 are provided in the kit, which are to be engaged upon the front and rear of the photo 24 sealing it therein from moisture and preventing UV light from damaging the underlying photo 24. The transparent sheets 25 may be of polyethylene or polyurethane or other plastic material adapted to block UV light from sunlight and protect the photo from moisture also. The adhesive is protected by a peel-and-stick type cover, or by engagement of one sheet 25 to the other prior to use.

The laminated photo 24 thereafter is stacked on the base 15 whereafter the cover sheet 22 is stacked to sandwich the photo 24 between the base 15 component and the cover sheet 22 component if employed. Thereafter the edge 16 is engaged around the perimeter of the stacked components placing all their respective perimeter edges within the slot 18 in a biased engagement between the two halves of the interior edge forming the window 20 which are pliable being formed of neoprene or rubber or other pliable but resilient material. The two half portions forming the slot 18 formed of plastic or rubber material, frictionally engage the stacked components around their opposing perimeters for a secure and moisture proof engagement. This yields the assembled device of FIG. 2 in the as-used mode, which may be worn by the user and which is substantially waterproof and provides a customized device 10 which each user may build from their own photos or indicia bearing materials. The placement of the indicia 23 on the templet formed by the cover sheet 22 is particularly preferred to identify the component to employ when trimming insertable photos or other indicia for the device 10. Users may be in an inhospitable country or may be unfamiliar with the device 10 but in a hurry and so identifying the cover sheet 22 as the templet 23 provides a means for quick identification of such to users. Positioning the templet 23 indicia at the edge where it will be hidden within the slot 18 is also preferred to eliminate the covering of photos or other materials by the lettering in the as-used mode. Finally, the cover sheet 22 may be formed of polarized material by using a laminated structure for the cover sheet 22 with a layer of horizontally polarizing material. This is also particularly preferred as it eliminates glare when the photo is viewed by the user in highly lit environments where glare can cause trouble viewing. Horizontal polarization is preferred as a vertical disposition of the polarization lines would cause the user to be unable to see the photo or picture behind the cover sheet 22 were they wearing polarized sun glasses.

While all of the fundamental characteristics and features of the user assembleable identification device and system herein, and employment thereof, have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the disclosed system and apparatus as defined by the following claims.

What is claimed is:

1. A user assembleable identification apparatus configured for wearing around the neck on a chain, comprising:
a kit of components assembleable to a readable tag, said components provided in an assembleable mode restrained on a chain, said chain providing means to maintain said components together prior to an assembly by said user;
said components including at least a malleable frame, a planar cover sheet, and a first protective sheet and a second protective sheet;
said malleable frame having an aperture surrounded at its perimeter by an edge portion of said frame;
said aperture having a slot formed in said edge portion;
said planar cover sheet having a circumference sized to engage within said slot;
said planar cover sheet sized to overlay upon a display sheet bearing indicia imprinted thereon and positioned adjacent thereto by said user and thereby provide a visual guide to cut said display sheet to a size to engage within said slot;
said first protective sheet and said second protective sheet both sized with a respective perimeter edge configured for an engagement for substantially all of said perimeter edge, within said slot;
said first protective cover sheet being substantially transparent and having an adhesive surface providing a means to engage in a mounted position upon a first side surface of said display sheet overlaying said indicia;
said second protective cover sheet having an adhesive surface providing a means to engage it to a mounted position upon a second side surface of said display sheet, opposite said first side surface;
said first and second protective cover sheets in said respective mounted positions providing means to prevent a contact of moisture with said display sheet; and
said display sheet, with said first and second cover sheets in said mounted positions, insertable to an engagement within said slot to form said identification apparatus in an as-used mode, wearable by said user as a means to identify said user and provide information concerning said user to third parties.

2. The user assembleable identification apparatus of claim 1, additionally comprising:
instruction indicia imparted upon said cover sheet, said instructions providing said user step by step assembly instructions to form said identification apparatus to said as-used mode.

3. The user assembleable identification apparatus of claim 1, additionally comprising:
said indicia imparted by said user includes one or a combination of indicia from a group of identification indicia including a photo of said user; a medical record associated with said user, and a bar code related in a database to personal or medical records of said user.

4. The user assembleable identification apparatus of claim 1, additionally comprising:
said instruction indicia including directions to said user to impart one or a combination of identification indicia, from a group of identification indicia including a photo of said user; a medical record associated with said user, and a bar code related in a database to personal or medical records of said user.

5. The user assembleable identification apparatus of claim 1, additionally comprising:
said first protective cover sheet having a horizontal polarization layer providing a means to enhance viewing of said identification indicia in bright or scattered lighting by eliminating glare reflected by said first side surface of said display sheet and concurrently allow a viewing of said identification indicia by a said third party wearing conventional polarized sunglasses.

6. The user assembleable identification apparatus of claim 2, additionally comprising:
said first protective cover sheet having a horizontal polarization layer providing a means to enhance viewing of said identification indicia in bright or scattered lighting by eliminating glare reflected by said first side surface of said display sheet and concurrently allow a viewing of said identification indicia by a said third party wearing conventional polarized sunglasses.

7. The user assembleable identification apparatus of claim 3, additionally comprising:
said first protective cover sheet having a horizontal polarization layer providing a means to enhance viewing of said identification indicia in bright or scattered lighting by eliminating glare reflected by said first side surface of said display sheet and concurrently allow a viewing of said identification indicia by a said third party wearing conventional polarized sunglasses.

8. The user assembleable identification apparatus of claim 4, additionally comprising:
said first protective cover sheet having a horizontal polarization layer providing a means to enhance viewing of said identification indicia in bright or scattered lighting by eliminating glare reflected by said first side surface of said display sheet and concurrently allow a viewing of said identification indicia by a said third party wearing conventional polarized sunglasses.
